(19) United States
(12) Patent Application Publication
(10) Pub. No.: US 2020/0306401 A1
(43) Pub. Date: Oct. 1, 2020

(54) ENCLOSED VESSEL SANITATION SYSTEM

(71) Applicant: TEKDRY INTERNATIONAL, INC., Lafayette, CO (US)

(72) Inventors: ADAM R. COOKSON, BROOMFIELD, CO (US); DANIEL STOREY, LONGMONT, CO (US)

(21) Appl. No.: 16/834,805

(22) Filed: Mar. 30, 2020

Related U.S. Application Data
(60) Provisional application No. 62/825,877, filed on Mar. 29, 2019.

(51) Int. Cl. A61L 2/24; F04D 15/00; A61L 2/04

(52) U.S. Cl. CPC A61L 2/24; A61L 2/04; F04D 15/0005

(57) ABSTRACT

A sterilization system and method are provided that convert a sterilant solution to a vapor using an outgassing process that removes sterilant vapor from a solution (e.g., liquid) or a solid and delivers the sterilant vapor into an interior of an enclosure vessel. The interior of the enclosed vessel is maintained at a vacuum pressure. Once the sterilant vapor enters the interior of the vessel, the vapor expands to all surfaces to effectively sterilizing the interior of the vessel.

### CROSS-REFERENCE

The present application claims the benefit of the filing date of U.S. Provisional Application No. 62/825,877 having a filing date of Mar. 29, 2019, the entirety of which is incorporated herein by reference.

### FIELD

The present disclosure relates to systems and methods for sterilizing large enclosures using vaporized sterilants. Such vaporized sterilants may include peroxide, such as hydrogen peroxide, or peroxy acid, such as performic acid. The presented systems and methods find particular application in the sterilization of tanks and/or vessels that are used in food, beverage and/or biopharmaceutical processing.

### BACKGROUND

Effective control of microbial contamination is paramount in ensuring the safety and quality of food/beverages and/or drugs. Microbial contamination is a significant concern in the production of widely consumed items such as beer/wine as well as pharmaceuticals/biopharmaceuticals. The complexity and methods by which brewing, and bioprocessing occur make these items especially susceptible to contamination from microorganisms.

A typical brewing and/or biopharmaceutical process is commonly performed in large stainless-steel tanks and/or vessels that are continually being exposed to desirable and undesirable microbes from the production process as well as from starting materials and/or from the surrounding environment. These enclosures can quickly become contaminated with unwanted microbes such as bacteria, molds, fungi, yeasts, spores, etc., especially in hard to reach spaces such as in small openings or crevices of the tanks and/or vessels and become difficult to eliminate. Surfaces soiled with microbes in this manner can become a source of contamination of and may make their way into a process stream of wort, beer or yeast of a food/beverage or the production, harvest, purification or formulation/fill with a biopharmaceutical. Contamination of beer or wine with undesirable yeast or bacterial affects the chemical composition and the performance of the brewing yeast causing adverse changes in acidity, aroma, turbidity and may render a batch unusable in turn wasting significant time, money and effort. Contamination of a pharmaceutical/biopharmaceutical with undesirable bacterial or fungal impurities, if severe, may cause patient morbidity and mortality. As such, processing equipment needs to be sterilized between each production batch to maintain product quality and safety and to keep cost and time commitments low.

In larger brewing facilities, a routine cleaning and sanitization procedure involves a clean-in-place (CIP) technique that is meant to cut the amount of work and make cleaning between batches useful and practical. A CIP technique involves utilizing a spray ball, located at the top of the tank and/or vessel, to distribute detergent and sanitizer throughout the enclosure. The detergent and sanitizer are circulated back through the tank and/or vessel after being drained at the bottom of the enclosure by a pump that lifts these components back up to the spray ball. In general, a cleaning process involves multiple steps, the first being a rinse and drain of plain water to remove excess debris.

The second step involves filling and circulating the tank and/or vessel with water that includes an alkaline cleaner (such that may consist of sodium hydroxide, sodium percarbonate, sodium carbonate, silicates, phosphate, etc.) at high temperatures. This step is meant to remove excess proteins and organic soils that have adhered to the surface of the enclosure. Multiple water rinses are performed following the detergent clean step to neutralize and to ensure removal of the basic treatment of chemicals/products. An acid rinse (such as a phosphoric acid mix) in cold or warm water follows the neutralization rinse and is meant to remove inorganic soil and further neutralizes caustic conditions in the tank and/or vessel. Following the acid rinse a cold-water rinse is performed to prepare for the sanitation procedure that utilizes water and a sterilant for killing microorganisms remaining in the enclosure. Common sterilants include acid-anionic surfactants, chlorine dioxide, iodophor, peroxyacetic acid, quaternary ammonium compounds, sodium hypochlorite, etc.). Even though processing pharmaceutical/biopharmaceuticals, it can be contemplated that similar processes are used for cleaning and sterilizing the tanks and/or vessels used for this purpose

The in-place cleaning system for industrial size processing tanks and/or vessels as described above have several disadvantages including the use of large amounts of water and potential exposure to high temperatures and/or harmful chemical cleaning/sterilization solutions during the cleaning and sanitization process. The systems and methods described herein provide an improvement over current methods used for sterilization of large or industrial scale tanks and/or vessels used for processing food/beverages and/or pharmaceuticals/biopharmaceuticals. Further, the disclosed systems and methods provide a more effective, low temperature and environmentally friendly sterilization process. Along these lines, the use of large amounts of water, harmful chemicals, and/or elevated temperatures may be avoided. Additionally, the sterilization process described herein provides a solution for achieving a more effective decontamination process for large enclosures that may contain intricate openings and spaces that may harbor hard to kill microorganisms.

### SUMMARY

It has been recognized by the inventors that exceptional sterilization of large enclosures, especially tanks and/or fermentation vessels used for food/beverage, pharmaceutical and/or biopharmaceutical processing can be achieved using a vaporized sterilant while the large enclosure is under vacuum pressure (e.g., below atmospheric pressure). That is, many such tanks or vessels are pressure vessels capable of holding positive pressure. These vessels are likewise capable of holding a vacuum pressure.

Further, it has been recognized that introducing a vaporized sterilant into an enclosure/vessel while the interior of that vessel is below atmospheric pressure allows the vaporized sterilant to expand all surfaces of the vessel. This allows sterilizing areas of such vessels that were hard to reach utilizing previous sterilization methodologies. The sterilant(s) may be introduced into the vessel in a vaporized form or may be introduced in a liquid form and allowed to vaporize in the low-pressure environment within the vessel. Suitable sterilants include, without limitation, peroxides, such as hydrogen peroxide, and/or peroxy acids, such as performic acid.

Generally, a sterilization system is provided that converts a sterilant solution to a vapor using an outgassing process that may deliberately and in a controlled manner remove sterilant vapor from a liquid solution or a solid and deliver the vapor into a large enclosure vessel to effectively sterilize interior surfaces of enclosure. In and arrangement, the system includes a reservoir configured to hold a supply of liquid or solid sterilant. The reservoir may include a heater to elevate the temperature of sterilant disposed therein. The reservoir is connectable to an interior of an enclosed vessel my a first fluid conduit. Typically, a valve in the fluid conduit permits opening (e.g., fluidly connecting) the reservoir to an interior of the vessel and isolating the reservoir from the interior of the interior of the vessel. The system further includes a depressurization system. The depressurization system is connectable to the interior of the enclosed vessel via a fluid conduit. In operation a pump (e.g., vacuum pump) of the depressurization system depressurizes the interior of the vessel to a desired vacuum pressure. Once depressurized, the valve connecting the reservoir to the interior of the enclosed vessel may be opened. The sterilant may outgas (e.g., vaporize) in the low-pressure environment. In a further arrangement, a sterilant pump may be used to assist in the delivery of sterilant to the interior of the enclosure. Such sterilant may be pumped in gaseous form or liquid form. In a further arrangement, a carrier gas may be supplied to assist in carrying the sterilant into the interior of the enclosure.

A method for sterilization of a large enclosure is also provided. The method comprises providing sterilant chamber that is adapted to receive a sterilant in the form of a liquid or solid and to convert the sterilant liquid into a vapor, under reduced pressure and/or heat. The vapor is directed into a vessel to be sterilized after an interior pressure of the vessel is reduced below an ambient atmospheric pressure. Sterilization of the tank and/or vessel is accomplished by circulating the sterilant vapors under reduced pressure through the tank and/or vessel using a vacuum apparatus with or without a carrier gas.

One advantage of the present disclosure is that it ensures that an enclosure, such as a food/beverage and/or bioprocessing tank and/or fermentation vessel, is free of microorganisms that may have adverse effects on the production of beer, wine or spirits and/or pharmaceutical/biopharmaceuticals.

Another advantage of the present disclosure is that the expansion of the vaporized sterilant under negative pressure enables the sterilization of small openings, crevices and other places within the enclosure that are hard to reach with liquid sterilants used in conventional techniques.

Another advantage of the present disclosure is that it enables sterilization of an enclosure, such as a food/beverage, bioprocessing tank and/or fermentation vessels, and the items contained within the enclosure to be sterilized in-place and to occur in a short period of time.

Another advantage of the present disclosure is that the sterilant vapors and their respective breakdown products are chemically compatible with a wide range of materials making them desirable for sterilizing food/beverage, bioprocessing and/or fermentation vessels and their inner components, such as plastic seals and/or glass windows.

Another advantage of the present disclosure is that it enables the sterilization of an enclosure, such as a food/beverage, bioprocessing and/or fermentation vessels, and the items contained within the enclosure to be sterilized without the use of large amounts of water, excessive heat and/or caustic and harmful sanitization chemicals. The sterilization process described in the invention may be performed at room temperature and/or at low temperatures.

Another advantage of the present disclosure is that it enables the sterilization of an enclosure, such as a food/beverage, bioprocessing and/or fermentation vessels, to occur while reducing the amount and type of residual sterilant chemicals remaining. The sterilization process described in this disclosure provides for the rapid degradation of the sterilant vapor molecules into innocuous components such as water, carbon dioxide, oxygen, etc.

### BRIEF DESCRIPTION OF THE FIGURES

FIG. 1 illustrates a first embodiment of a sterilization system.

FIG. 2 illustrates a second embodiment of a sterilization system.

### DETAILED DESCRIPTION

Reference will now be made to the accompanying drawing, which at least assists in illustrating the various pertinent features of the presented inventions. The following description is presented for purposes of illustration and description and is not intended to limit the inventions to the forms disclosed herein. Consequently, variations and modifications commensurate with the following teachings, and skill and knowledge of the relevant art, are within the scope of the presented inventions. The embodiments described herein are further intended to explain the best modes known of practicing the inventions and to enable others skilled in the art to utilize the inventions in such, or other embodiments and with various modifications required by the particular application(s) or use(s) of the presented inventions.

Provided herein are systems and methods for sterilizing an enclosure such as food/beverage vats, bioprocessing tanks, and/or fermentation vessels (hereafter ‘vessel’ or ‘vessels’). The system and methods may be utilized to sterilize or sanitize any vessel that is capable of holding and maintaining a negative or vacuum pressure (i.e., a pressure below ambient atmospheric pressure). Such vessels tend to have various openings or ports (e.g., fluid ports) such as exhaust ports and drains that can be utilized with the systems and methods described herein. The systems and methods introduce a sterilant into such vessels while an interior of the vessel is below ambient atmospheric pressure. The sterilant, which is pre-vaporized or vaporizes within the enclosed vessel, expands to reach all interior surfaces of the enclosed vessel. In an embodiment, the systems and methods make use of a peroxide, such as hydrogen peroxide, and/or a peroxy acid, such as performic acid, vapor to sterilize an enclosed vessel. Other vapors that are capable of eliminating target microorganisms may also be used with the systems and methods.

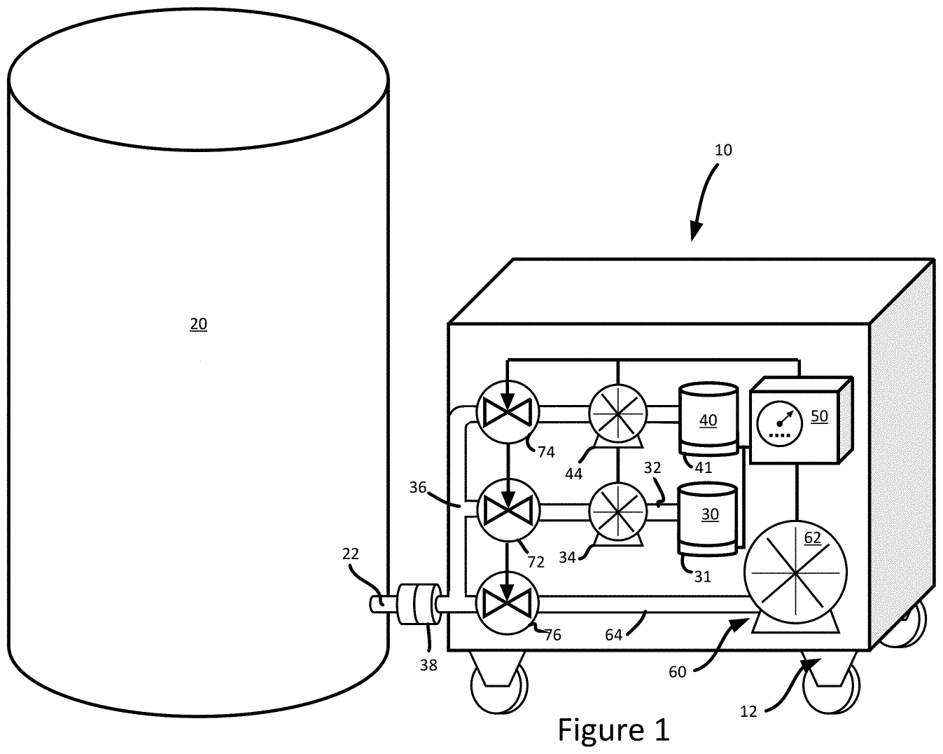

FIG. 1 illustrates one exemplary enclosed vessel sterilization system 10 that is configured to sterilize or sanitize an enclosed vessel 20. The system 10 include a sterilant chamber or reservoir 30 that may sized based on the size of the enclosed vessel 20 that will be serviced. Most commonly, the reservoir 30 holds a sterilant in liquid form though the reservoir may alternately hold solid form sterilants. The system 10 also includes a depressurization system 60 that is utilized to reduce the pressure within the enclosed vessel 20 to a predetermined pressure below ambient atmospheric pressure. The system may also include an optional carrier gas system 40, which may be used to facilitate introduction of the sterilant into the interior of the enclosed vessel 20.

The reservoir 30 is connected to a fluid conduit 32 which may be fluidly connected to the interior of the enclosure. In the illustrated embodiment, the fluid conduit 32 connects to a manifold 36. The manifold includes an inlet coupling 38 that is connectable to a port 22 of the enclosure 20. A valve 72 is disposed within the fluid conduit 32 to selectively open a fluid path between the reservoir 30 and the manifold 36 and, hence, the interior of the enclosed vessel. The valve 72 may be may be manually operated or connected to a system controller 50. In the latter regard, the valve 72 includes an actuator permitting operation of the valve based on control signals from the controller 50. The valve 72, when under operation manually or by automation may, in one embodiment, serve to control a vacuum level inside of the reservoir thereby controlling a rate of outgassing or formation and supply of a sterilant vapor to the interior of the vessel vessel 2. In such an embodiment, the interior of the vessel may be depressurized prior to opening the valve 72. Once opened, liquid sterilant or solid form sterilant within the reservoir is exposed to low pressure and begins vaporizing. A predetermined volume of sterilant may be disposed in the reservoir to provide a predetermined volume of sterilant vapor to the interior of the enclosed vessel.

The sterilant chamber 30 may also be equipped with a mixing device for stirring or mixing liquid sterilant and/or sterilant components. Such mixing or stirring may occur manually or may be automated. In such an arrangement, two or more sterilant components may be placed or injected into the reservoir 30 such that they may be mixed at or near the time of use. The sterilant chamber may also be equipped with a heater 31 (e.g., resistive, radiant, etc.) to facilitate vaporization of the sterilant. Though not required, heat may be applied to the sterilant chamber and/or any part(s) of the assembly described above by radiation and conduction and may serve to improve the efficiency of sterilant vapor outgassing and mobility of sterilant vapor and/or carrier gas molecules throughout the enclosure.

Sterilant may be placed directly inside of the sterilant chamber by means of accessing the reservoir using a latch or handle or the sterilant liquid may be delivered to the sterilant chamber from a larger external sterilant storage cartridge by means of an automated syringe, injector. In such an arrangement, a delivery pump(s) (not shown) may be utilized to deliver sterilant and/or sterilant components to the reservoir. Such flow into the reservoir may be regulated by a liquid flow controller and meter. That is, a liquid flow controller and meter may be used to regulate the amount of sterilant liquid introduced into the chamber. This may be useful, for example, in a situation when a more substantial amount of sterilant liquid is required to sterilize a tank or vessel known to contain a larger bio-load or more resistant microbes.

A liquid flow controller and meter may also be used to regulate the amount and type of sterilant liquid components that are introduced into the reservoir. This may be used to regulate the sterilant liquid formulation and/or rate of formation. For example, a higher purity peroxy acid solution may be formed faster by appropriately adjusting the parent component liquid volume ratios that are injected into the sterilant chamber. The syringe or delivery pump is connected to the sterilant chamber by way of conduit(s) or tube(s) that introduce the sterilant liquid or sterilant liquid components into the reservoir of the sterilant chamber.

In some instances, the sterilant from the reservoir 30 may be pumped directed into the enclosure 20. In such an arrangement, a sterilant pump 34 may be disposed proximate to the reservoir 30. For instance, the sterilant pump 34 (e.g., a liquid flow controller and meter) may be disposed within he fluid conduit 32 between the reservoir 30 and the valve 72. The sterilant pump 34 may be configured to pump liquid from the reservoir into the manifold and, hence, the interior of the enclosed vessel where the liquid sterilant may evaporate/vaporize in the low-pressure environment. Alternatively, the sterilant pump may pump a gaseous sterilant from the reservoir into the interior of the enclosed vessel 20. In a further embodiment, the sterilant pump may be a vacuum pump that is configured to apply a vacuum to the reservoir. In such an arrangement, the vacuum sterilant pump 30 may exhaust vaporized sterilant into the fluid conduit which may migrate to the interior of the enclosure. In any pumping arrangement, the pump may include a flow meter to allow determining a volume of sterilant supplied to the interior of the enclosure.

To facilitate movement of the sterilant (e.g., liquid or vaporized) a carrier gas, such as purified air from, for example, a carrier gas reservoir 40 may be supplied to the fluid path of the sterilant. In an embodiment, a carrier gas pump 44 supplies a carrier gas to the manifold 38 upon the opening of a carrier gas valve 74. Once this valve 74 is opened, the carrier gas mixes with the sterilant liquid or mix with the sterilant vapor and help mobilize the sterilant into the enclosure 20. The purified air is supplied to the sterilant chamber from a device, such as a compressor or a pressurized gas cylinder, by way of a gas conduit or line. The carrier gas may be purified or filtered before entering the sterilant chamber. It also may be dehumidified or humidified before entering the sterilant chamber. Additionally, the carrier gas may be heated before mixing with the sterilant chamber connected to the carrier gas reservoir. In an embodiment, a heater 42 (e.g., resistive, radiant, etc.) may be attached to the gas reservoir. In some instances, the carrier gas may be supplied directly to the enclosed vessel instead of or in addition to being supplied to the sterilant prior to entering the enclosed vessel.

As noted above, the system includes depressurization system 60 that is utilized to reduce the pressure within the enclosed vessel 20 to a predetermined pressure below ambient atmospheric pressure. The main component of the depressurization system is a dry scroll vacuum pump 62 or equivalent that can be operated manually or operated by the controller 50 (e.g., automated). In the illustrated embodiment, the pump 62 (e.g., vacuum pump) attaches to the enclosed vessel 20 via a fluid conduit 64 and a valve 76. In this embodiment, the sterilant reservoir 30, carrier gas reservoir 40 and depressurization system 60 all connect to a common manifold 38 that attaches to a single port of the enclosed vessel. However, this is not a requirement. For instance, the vacuum pump 62 may connect to a separate port of the enclosed vessel by way of a separate vacuum line (e.g., rigid or flexible). In any embodiment, air is evacuated from inside of the enclosure 20 using the vacuum pump 62 upon the start of the sterilization process. If the vacuum pump and the sterilant reservoir 30 connect to the interior of the pressure vessel via different ports, operation of these components may at least partially overlap. Any unused sterilant vapor left in the enclosed vessel may likewise be removed utilizing the depressurization system. Such remaining sterilant may undergo an additional degradation and/or absorption step to prevent sterilant vapor from being released into the environment. This may be accomplished by passing the residual sterilant vapor through a catalytic converter, an absorptive filter/material, a trap and/or a plasma.

The operational components of the system, such as the valves, pumps, heaters, and the like that are automated may be controlled by a controller 50 (e.g., processing unit. Sensors and detectors may be linked to the central processing unit to provide feedback of system operations such as sterilant vapor concentration, humidity, vacuum pressure, temperature. By employing a feedback system such as that described in this disclosure the components of the system can be controlled to maintain desired ranges (e.g., sterilant vapor concentration) and/or respond to system failures.

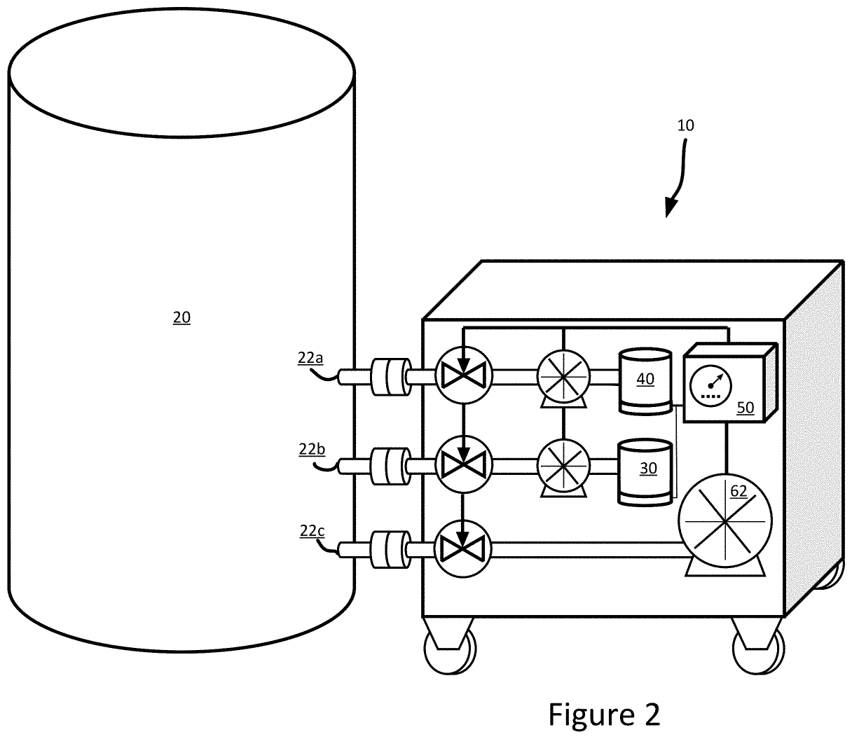

The system 10 may be affixed to a mobile cart 12 that can easily be transported from one place to another. When larger or multiple enclosures need to be sterilized, various initiation systems may be used. Though the system 10 is illustrated in FIG. 1 as having a single manifold attached to a single port 22 of the enclosed vessel, it will be appreciated that system components (e.g., 30, 40, 60) may attached to separate ports 22a, 22b, 22c as illustrated in FIG. 2.

The enclosure 20 to be sterilized may include surfaces that are commonly used when processing beer, wine, liquors and spirits, pharmaceuticals and/or biopharmaceuticals, such as stainless steel, though any material surface may be sterilized whether metal, plastic or ceramic. The size of the enclosures to be sterilized using the method described herein may range between 15 and 300,000 liters, though any size enclosure may be sterilized. It may be appreciated that while particular reference is made to the sterilization of food/beverage and/or bioprocessing tanks and/or fermentation vessels, the sterilization system described herein has application in other enclosures that are capable of maintaining pressures below ambient atmospheric pressure.

An exemplary enclosure sterilization process occurs as described, although other permutations of the process are considered. Initially, an operator(s) may position the system near tan enclosure vessel to be sterilized. Before initiation the sterilization process, the brew tank and/or fermentation vessels (or similar vessel) will have been rinsed with water using a high-pressure hose to remove solid particulate remaining in enclosure following a production run. The operator connects and locks the vacuum line to the exhaust port of the vessel. The operator begins the sterilization run. This may entail launching the process on the control system at which point the actions of the sterilization system may be automated by the central processing unit. Effects of the sterilization system process occur as described below. The sterilant chamber and components may be heated if required. The enclosure vessel may be heated if required. Once the system reaches the temperature of the selected process, a valve between the sterilant supply and the sterilant chamber opens, and the sterilant liquid is transferred from sterilant supply into the sterilant chamber for processing.

Vacuum is initiated using the depressurization system to bring the enclosed vessel to a set base pressure. Such a base pressure is sufficient enough to reduce the humidity level of the enclosure to a desired level. A valve between the depressurization pump and the enclosure may be closed. At this time, a valve between the sterilant chamber and vessel opens. This allows outgassing of the sterilant liquid within the sterilant chamber to occur under reduced pressure. Sterilant liquid vapor travels into and fills the tank and/or vessel by manipulating the vacuum effectively sterilizing the surface of the enclosure. At run completion, excess sterilant vapor is degraded, and the enclosure is brought to atmosphere ready for future product processing.

Sterilization is used herein to include the eradication of biological contaminants, especially microorganisms which include bacteria, bacterial spores, viruses, molds, and fungi. Sterilization, the top-level control of contamination by microorganisms, has been defined by The Association for the Advancement of Medical Instrumentation (AAMI) as: “A process designed to remove or destroy all viable forms of microbial life, including bacterial spores, to achieve an acceptable sterility assurance level.” Sterility is measured by probability expressed as sterility assurance level (SAL) and is generally accepted that a SAL of 10-6. A SAL of 10-6 means that there is less than or equal to one chance in a million that a particular item is contaminated or unsterile following a sterilization process.

Example microorganisms responsible for contamination of enclosures used to prepare beer, wine and/or spirits include: bacteria such as those belonging to the genera Acetobacter, Gluconobacter, Lactobacillus, Lenconostoc, Pediococcus, Zymomonas spp., Pectinatus spp., and/or various Enterobacteriaceae; species and strains of yeast including Brettanomyces, Candida, Hanseniaspora, Pichia and/or Zygosaccharomyces; single spore and large cell aggregate mold belonging to the genera Mucor, Penicillium, Aspergillus, Cladosporium, Geotrichum, and/or Rhizopus. Example microorganisms responsible for contamination of enclosures used for bioprocessing include: bacteria such as those belonging to the genera Staphylococcus, Streptococcus, Micrococcus, Corynebacterium, Actinomyces, Arthrobacter, Bacillus, Clostridium, Pseudomonas, Ralstonia, Burkholderia, Acinetobacter, Stenotrophomonas, Serratia, and/or Klebsiella; yeasts such as those belonging to the genera Candida, Rhodotorula and/or Cryptococcus; single spore and large cell aggregate mold belong to the genera Aspergillus, Penicillium, Alternaria, Fusarium and/or Cladosporium; Mycoplasma hyorhinis, Mycoplasma arginine, Mycoplasma orale and/or Acholeplasma laidlawii; viruses such as Bacteriophage, Parvovirus, Retrovirus and/or Bovine Viral Diarrhea Virus.

Some embodiments described herein use hydrogen peroxide vapor is an effective sterilant due to its broad-spectrum action against an extensive range of microorganisms, especially those hard to destroy spore species such as Geobacillus stearothermophilus. This sterilant vapor is particularly useful for this invention because it is potent at low concentrations and at low temperatures, including at room temperature. The molecules and their breakdown products have good material compatibility and are safe to use with a variety of equipment.

Some embodiments described herein use performic acid as the peroxy acid of choice due to its high vapor pressure and tendency to volatilize rapidly, although there are others that will suffice. For example, performic acid has a higher vapor pressure (78 mmHg @25° C.) over its commonly used counterpart peracetic acid (14.5 mmHg @25° C.), making it more readily able to vaporize, allowing for an accelerated sterilization run. By the same token, performic acid is firstly vaporized from a mixture of its parent components, formic acid, and aqueous hydrogen peroxide, making it an attractive choice for selectively excluding undesirable residual components, such as water vapor or formic acid, during a sterilization process. Additionally, performic acid is non-toxic, and there are no reports of tumorigenic properties. The bactericidal and sporicidal properties of peracetic acid, performic acid, and perpropionic acids have been documented, and of the three peroxy acids, performic acid activity as an anti-fungicide surpasses that of its counterparts. Performic acid's mode of action, as an active oxidizing agent for killing microbial cells, is a highly effective and fast-acting process of cleaving disulfide bonds of microbial cells ultimately causing the death of the cell. In addition to performic acid's highly effective kill mechanism, the sterilization action of performic acid is faster and has a lower concentration threshold than that of related peroxy acid compounds such as peracetic acid. The Association of Official Analytical Chemists (AOAC) completed a Sporicidal Challenge and found that the D-value of performic acid may be lower than five minutes at a low concentration of 1,800 ppm at 44° C.

### EMBODIMENTS

### Peroxide and Peroxy Acid Solutions

### Peroxide Solution

According to an embodiment, a peroxide solution can be generated in a way that compliments the need of the user. For example, the formulation of the peroxide solution can be adjusted according to its intended use. It may contain a more pure and concentrated solution if it is to be used for multiple sterilization runs, for killing more resistant microbes and/or for sterilizing a larger bio-load. As such, peroxide solution chemistries described herein are meant to include mixed peroxide chemistries. The formulation methods described may or may not utilize a catalyst to accelerate the formation of the peroxide.

Additionally, the formulation methods described may or may not utilize a stabilizer to prevent reaction and subsequent decomposition enhancing the shelf-life and safety of the sterilant solution. The selective production of peroxide can be achieved by controlling the type and proportions of component chemicals as well as reaction conditions, such as temperature and rate of mixing. The average peroxide content is preferably between 30-50 weight % however the average concentration may vary. In some embodiments, the peroxide compositions may be generated from a mixture of more than one peroxide or source of peroxide.

### Peroxide Components

In an embodiment, the peroxide includes an aqueous hydrogen peroxide solution that can be introduced into the sterilant chamber at a concentration between 1 and 90 weight %, more preferably at a concentration between 30 and 50 weight %. The peroxide solution may be diluted by a solvent before being introduced into the sterilant chamber or after being introduced into the sterilant chamber.

In another embodiment the peroxide can be generated by reacting alternative peroxides and/or alkali metal peroxide salts or combinations of that include urea-hydrogen peroxides, hydrogen peroxide donors of group 1 (IA) (e.g. lithium peroxide, sodium peroxide), of group 2 (IIA) (e.g. magnesium peroxide, calcium peroxide, strontium peroxide, barium peroxide), of group 12 (IIB) (e.g. zinc peroxide), of group 13 (IIIA) (e.g. sodium perborate hexahydrate, sodium peroxyborate tetrahydrate, sodium perborate monohydrate), of group 14 (IVA) (e.g. persilicates, peroxycarbonates), of group 15 (VA) (e.g. peroxynitrous acid, peroxyphosphoric acid), of group 16 (VIA) (e.g. peroxysulfuric acid, peroxydisulfuric acid) and group VIIa (e.g. sodium periodate, potassium perchlorate), and transition metal peroxides. The alternative peroxide source may consist of a liquid or a solid and may be used in the methods described in this invention. Any suitable substance that generates peroxide at a concentration between 1 and 90 weight % may be used. Stabilizing agents may be added to stabilize the peroxide solution and prevents decomposition. Example stabilizers are further described below.

### Peroxyacid Solution

According to an embodiment, a peroxy acid solution can be generated in a way that compliments the need of the user. For example, the formulation of the peroxy acid solution can be adjusted according to its intended use. It may contain a more pure and concentrated solution if it is to be used for multiple sterilization runs, for killing more resistant microbes and/or for sterilizing a larger bio-load. As such, peroxy acid solution chemistries described herein are meant to include mixed peroxy acid chemistries. The formulation methods described may or may not utilize a catalyst to accelerate the formation of the peroxy acid.

Additionally, the formulation methods described may or may not utilize a stabilizer to prevent reaction and subsequent decomposition enhancing the shelf-life and safety of the sterilant solution. The selective production of peroxy acid can be achieved by controlling the type and proportions of component chemicals as well as reaction conditions, such as temperature and rate of mixing. The average peroxy acid content is preferably between 0.5 and 20 weight %, however, the average concentration may vary depending on reaction condition variables. In some embodiments, the peroxy acid compositions may be generated from a mixture of more than one peroxy acid.

### Peroxy Acid Components

In one embodiment, the peroxy acid can be prepared by mixing one or more carboxylic acids followed by the addition of a hydrogen peroxide solution. A preferable concentration of carboxylic acid is introduced into the reaction mixture may be between 25 and 100 weight %. The hydrogen peroxide concentration may be between 1 and 90 weight % and may be introduced into the reaction mixture at a preferable concentration between 5 and 60 weight %. The carboxylic acids used for formation of the peroxy acid components may consist of an aliphatic carboxylic acid that may include but is not limited to acetic acid, formic acid, propionic acid, oxalic acid, malic acid, maleic acid and fumaric acid or mixtures of such. The carboxylic acids used for formation of the peroxy acid components may also consist of an aromatic carboxylic acid that may include but is not limited to benzoic acid, salicylic acid, gallic acid, toluic acid, phthalic acid, isophthalic acid, and terephthalic acid.

In another embodiment, the peroxy acid can be generated by reacting an alternative carboxylic acid source with a reactive oxidizing species. The alternative carboxylic acid source may consist of a liquid or a solid and may be used in the methods described in this invention. Any suitable substance that is a peroxy acid generator may be used. The peroxy acid source may consist of one or more salt of the desired carboxylic acid, for example, the salt of acetic acid, such as an acetate, e.g., sodium or ammonium acetate. Additionally, the carboxylic acid source may also consist of one or more ester alcohol(s), such as ethyl acetate and/or propyl acetate. Any combination of salt(s) and alcohol(s) may also be utilized for this purpose. The reactive oxidizing species may include hydrogen peroxide or a hydrogen peroxide derivative that delivers a hydrogen peroxide concentration between 1 and 90 weight % of hydrogen peroxide. Suitable species may consist of one or more of the following alternative peroxides and/or alkali metal peroxide salts or combinations of that include urea-hydrogen peroxides, hydrogen peroxide donors of group 1 (IA) (e.g. lithium peroxide, sodium peroxide), of group 2 (IIA) (e.g. magnesium peroxide, calcium peroxide, strontium peroxide, barium peroxide), of group 12 (IIB) (e.g. zinc peroxide), of group 13 (IIIA) (e.g. sodium perborate hexahydrate, sodium peroxyborate tetrahydrate, sodium perborate monohydrate), of group 14 (IVA) (e.g. persilicates, peroxycarbonates), of group 15 (VA) (e.g. peroxynitrous acid, peroxyphosphoric acid), of group 16 (VIA) (e.g. peroxysulfuric acid, peroxydisulfuric acid) and group VIIa (e.g. sodium periodate, potassium perchlorate), and transition metal peroxides.

The same may be reacted in the presence of a catalyst to accelerate the formation of the peroxy acid. Commonly used catalysts include but are not limited to mineral acids such as sulfuric acid, phosphoric acid, pyrophosphoric acid and/or polyphosphoric acid.

One or more catalysts can be added to the reaction mixture in a concentration ranging from between 0.01 and 20 weight %. The catalyst may be added to a peroxy acid solution. Alternatively, the catalyst may be dissolved independently in either the hydrogen peroxide or the carboxylic acid before mixing. Or, the catalyst may be dissolved in another suitable solvent before adding to the mixture of mixture components. Stabilizing agents may be added to stabilize the peroxide solution and prevents decomposition. Example stabilizers are further described below.

### Stabilizers

In some embodiments, a stabilizing agent is preferred for certain peroxide or peroxy acid compositions. In such cases, one or more stabilizers may be added to prevent reaction and subsequent decomposition all while enhancing the shelf-life and safety of the peroxide or peroxy acid solution. Stabilizers are selected that do not adversely alter the vapor pressure or the purity of the active ingredient in the sterilant solution. Equally important, stabilizers are chosen such that they may not counteract the vaporization properties and the anti-microbial properties of the peroxide or the peroxy acid. One or more agents can be used and may include, but is not limited to, amino- or hydroxyl-polyphosphonic acid or soluble salt, polymeric carboxylic acid or soluble salt, hydroxycarboxylic acids, aminocarboxylic acids, heterocyclic carboxylic acids (e.g. dipicolinic acid), 1-hydroxyethylidene-1,1-diphosphonic acid (HEDP), other phosphonic acids and phosphonate salts such as ethylenediamine tetrakis methylenephosphonic acid (EDTMP), diethylenetriamine pentakis methylenephosphonic acid (DTPMP), cyclohexane-1,2-tetramethylene phosphonic acid, amino[tri(methylenephosphonic acid)], ethylenediamine[tetra methylene-phosphonic acid], 2-phosphene butane-1,2,4-tricarboxylic acid, other salts such as the alkali metal salts (e.g. ammonium or alkyloyl amine salts), mono-, di-, or tri- ethanolamine salts, picolinic acid or combinations thereof.

Other examples of compounds that have been used in solutions to protect against decomposition include but are not limited to sodium phenol sulfate, sodium stannate, alkyl anilines, sulfaimc acid sulfolane, alkyl sulfones and sulfoxides, phosphonic acids and their salts, acrylic acid polymers, polyphosphates, polyamino polyphosphonic acids and/or their salts, mineral acids (e.g. sulfuric acid, phosphoric acid, hydrochloric acid) and/or colloidal silicates. The stabilizer may be added to a mixture of peroxides and/or peroxy acids or a mixture of peroxide and/or peroxy acid and a catalyst. Alternatively, the stabilizer may be dissolved independently in either the components chemicals of the peroxide and/or peroxy acid solution(s). Or the stabilizer may be dissolved in another suitable solvent before adding to the mixture or the mixture components.

### Peroxide or Peroxy Acid Solution Stabilization Matrix

In another embodiment, peroxide or peroxy acid is vaporized, preferably, from a liquid containing a peroxide and/or peroxy acid solution that may comprise one or more peroxide(s) and/or one or more peroxy acid(s) that are encapsulated in a stabilization matrix. The stabilizing matrix that contains the peroxide or the peroxy acid is called the sterilant matrix and is preferably placed in the sterilant chamber, though in some instances it may be placed directly in the tank and/or vessel, to undergo the sterilization process in which the tank and/or vessel is pressurized to a vacuum level sufficient to gasify the peroxide or peroxy acid solution within the sterilant matrix. The peroxide or peroxy acid solution can be stabilized in a material matrix. It is in this material matrix that the peroxide or the peroxy acid solution molecules are reversibly trapped in the interior of the matrix to prevent reaction and to suppress degradation. The stabilization matrix may consist of but is not limited to, synthetic polymers, biopolymers, ceramics, glass, or composite materials, or any combination of these materials. Some examples of synthetic polymers that may be used include, but are not limited to, silicones, polyacrylates, polyethylenes, and related polymers, polyamides, polyurethanes, polyethers, polyphosphazenes, polyanhydrides, polyacetals, poly(ortho esters), polyphosphoesters, polycaprolactones, polylactides, and polycarbonates. Some example of biopolymers that may be used include, but are not limited to, carbohydrates, starches, celluloses, chitosans, chitins, dextrans, lignins, and polyamino acids. Some examples of ceramics include, but are not limited to, aluminum oxide, zirconium oxide, silicon dioxide, magnesium oxide, titanium oxide, aluminum nitride, silicon nitride, boron nitride, and silicon carbide. Composite material may consist of but are not limited to two or more of the matrix materials listed above.

The peroxide or peroxy acid solution may be prepared in liquid form using any of the methods previously described in this disclosure then immediately mixed or loaded into the stabilization matrix. The sterilant matrix is used to form a gel or a solid that contains the peroxide or the peroxy acid molecules. The sterilant gel or solid may be shaped into the form of beads or a block and/or may be stored in a container or wrapping that is sufficiently porous and such that allows the peroxide or the peroxy acid vapor molecules to outgas from the material matrix and/or container when exposed to reduced pressure and/or increased temperature. The purity and concentration of peroxide or peroxy acid loaded into the material matrix can be adjusted according to the intended use. It may be seen that a sterilant gel or solid that contain a purer and more concentrated form of peroxide or peroxy acid may be used for multiple sterilization runs, for killing more resistant microbes, and/or for sterilizing a larger bio-load. The sterilizing system can be configured for a user to quickly and safely place the sterilant gel or solid into the chamber before pressurizing the system and before initiating a sterilization cycle. This embodiment also provides a simplified method for extending the shelf-life of the peroxide or peroxy acid in such a way that the sterilant does not need to be prepared just before use. It may sit at a reasonable temperature without spontaneously degrading for an extended period giving the user the opportunity to pull the sterilant gel or solid off of the shelf when needed to insert it into the system for one or more uses. When contained in a stabilization matrix, peroxide or peroxy acid can safely be produced in bulk for use within sterilizing systems, such as those described herein.

### Packaging and Delivery Device

### Ampoule System

In another embodiment, the peroxide or the peroxy acid chemicals or components chemicals (e.g. carboxylic acid, and/or carboxylic acid source, hydrogen peroxide and/or reactive oxygen species, catalyst, stabilizer, diluent solvent, etc.) are packaged separately as part of a multi-component ampoule system and is used for preparing an active peroxide or peroxy acid liquid solution just prior to use in the sterilant chamber. In one aspect of the embodiment, the component chemicals can first be mixed inside the ampoule or ampoule housing and then second, can be injected or dispersed into a chemical mixing apparatus of the sterilant chamber, or directly into the tank and/or vessel itself. In a second aspect of this embodiment, the component chemicals can be mixed outside of the ampoule or ampoule housing after the isolated chemicals are injected or dispersed into a chemical mixing apparatus of the sterilant chamber or directly into the tank and/or vessel itself. The chemical mixing apparatus of the sterilant chamber is configured to receive a multi-component ampoule in such a way that the ampoule, at the proximal end, is locked into a port of the system. The proximal end of the ampoule that is locked into the system contains a membrane that is capable of being pierced by a needle or a cannula and a connector, such as a lure lock, that locks the ampoule housing into the system. The pierceable membrane of the ampoule seals off the contents of the ampoule from the system until pierced by a needle or other such device of the chemical mixing apparatus. Once penetrated, the mixed or isolated chemicals of the ampoule are injected or dispersed into the system for further mixing, heating and/or vaporization. Located distally from the lock assembly and the membrane is the ampoule housing that may be in the shape of a cylinder. The ampoule housing is made of a thermoplastic known in the art. Within the ampoule housing is a compartment that may be in the shape of a tube. This tube is made of glass that extends internally into the ampoule housing from the distal end of the ampoule. There may be one or more compartments per ampoule housing. Each compartment is a separate storage unit and is designed to hold a volume of liquid or solid chemical. The volume of chemical that each compartment can hold can be altered by increasing or decreasing the size of the compartment and/or the size of the ampoule housing. The compartment is attached to a flange at the distal end of the ampoule housing and is sealed by a plug. The separation of ampoule housing from the compartment or compartments are meant to keep the chemicals stored until needed by the user.

### Cartridge System

Another embodiment may contain separate cartridges that are used to store the component chemicals of the peroxide and/or the peroxy acid solution. It can be envisioned that if the user required, a peroxide sterilant solution, the component chemicals, one or more peroxide or other reactive oxygen species solutions, a diluent solvent, and/or a stabilizer, are stored separately in individual cartridges near the tank and/or vessel. It can also be envisioned that if the user required a peroxy acid sterilant solution, the component chemicals, one or more carboxylic acid and/or carboxylic acid source, hydrogen peroxide and/or reactive oxygen species, diluent solvent, catalyst and/or stabilizer, are stored separately in individual cartridges near the tank and/or vessel. The cartridges are designed to hold a volume of liquid and may be sealed under vacuum. The cartridges are designed to plug directly into the side of the sterilant chamber or into the tank and/or vessel. The sterilization system is configured to receive these cartridges in such a way that the cartridges, at the proximal end, are locked into the system. The proximal end of the cartridge that is locked into the system contains a membrane that is capable of being pierced. The pierceable membrane of the cartridge seals off the contents of the cartridge from the system until pierced by a needle or cannula of the sterilant chamber or the tank and/or vessel. It can also be envisioned that the cartridges are attached to the sterilant chamber or the tank and/or vessel by way of injection tubing that is connected to an injection pump controlled by a liquid flow controller. Chemicals stored in each of the cartridges are injected or dispersed into the sterilant chamber or the tank/or vessel where the chemical components are mixed, placed under reduced pressure and/or heated to form sterilant vapor. The system can be programmed to withdraw any volume of any chemical from each cartridge to generate the desired amount and concentration of sterilant solution. The separation of component chemicals in cartridges are meant to keep the chemicals stored until just before use safely. Also, this embodiment provides a method for safely generating a sterilant liquid vapor source at higher concentrations for shorter sterilization run times, if need be, as mixing of the component chemicals followed by controlled heating and vaporization of the sterilant will be performed under reduced pressure in a vacuum system.

### EXAMPLES

### Example 1

A peroxide solution consisting of hydrogen peroxide, 50 weight % (Sigma Aldrich, St. Louis, Mo.) was placed in the sterilant chamber and was tested for its microbicidal effectiveness against Geobacillus stearothermophilus spores in a small fermentation vessel using the system described in the disclosure. Multiple self-contained biological spore ampoules (Mesa Laboratories, Bozeman, Mont.) containing a stainless steel disc inoculated with a spore population of 106 spores/unit were placed at multiple locations within the enclosure. The enclosure was evacuated to a base pressure of 0.01 Torr. After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 2 Torr for 15 minutes after which point the chamber was brought to atmosphere with argon gas. The spore ampoules were removed from the enclosure using well-established sterile techniques. The stainless steel discs were tested for SAL by gently crushing the ampoule and then incubating the ampoule for 24 hours at 60° C. for 24 hours. After a time, it was found that the discs achieved an acceptable SAL of 10-6 spores/unit.

### Example 2

A peroxide solution consisting of hydrogen peroxide, 50 weight % (Sigma Aldrich, St. Louis, Mo.) was placed in the sterilant chamber and was tested for its microbicidal effectiveness against Geobacillus stearothermophilus spores in a large fermentation vessel using the system described in the disclosure. Multiple self-contained biological spore ampoules (Mesa Laboratories, Bozeman, Mont.) containing a stainless-steel disc inoculated with a spore population of 106 spores/unit were placed at multiple locations within the enclosure. The enclosure was evacuated to a base pressure of 1 Torr.

After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 20 Torr for 120 minutes after which point the chamber was brought to atmosphere with argon gas. The spore ampoules were removed from the enclosure using well-established sterile techniques. The stainless steel discs were tested for SAL by gently crushing the ampoule and then incubating the ampoule for 24 hours at 60° C. for 24 hours. After a time, it was found that the discs achieved an acceptable SAL of 10-6 spores/unit.

### Example 3

A performic acid sterilant solution was generated by mixing an aqueous solution of hydrogen peroxide, 30 weight % (Sigma Aldrich, St. Louis, Mo.) with formic acid, 98-100% (Sigma Aldrich, St. Louis, Mo.). The performic acid sterilant solution was stirred at 200 RPM for 15 minutes.

The performic acid sterilant vapor was tested for its microbicidal effectiveness against Geobacillus stearothermophilus spores in a small fermentation vessel using the system described in the disclosure. Multiple self-contained biological spore ampoules (Mesa Laboratories, Bozeman, Mont.) containing a stainless-steel disc inoculated with a spore population of 106 spores/unit were placed at multiple locations within the enclosure. The enclosure was evacuated to a base pressure of 5 Torr. After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 100 Torr for 15 minutes after which point the chamber was brought to atmosphere with argon gas. The spore ampoules were removed from the enclosure using well-established sterile techniques. The stainless-steel discs were tested for SAL by gently crushing the ampoule and then incubating the ampoule for 24 hours at 60° C. for 24 hours. After a time, it was found that the discs achieved an acceptable SAL of 10-6 spores/unit.

### Example 4

A performic acid sterilant solution was generated by mixing an aqueous solution of hydrogen peroxide, 30 weight % (Sigma Aldrich, St. Louis, Mo.) with formic acid, 98-100% (Sigma Aldrich, St. Louis, Mo.). The performic acid sterilant solution was stirred at 200 RPM for 15 minutes.

The performic acid sterilant vapor was tested for its microbicidal effectiveness against Geobacillus stearothermophilus spores in a large fermentation vessel using the system described in the disclosure. Multiple self-contained biological spore ampoules (Mesa Laboratories, Bozeman, Mont.) containing a stainless-steel disc inoculated with a spore population of 106 spores/unit were placed at multiple locations within the enclosure. The enclosure was evacuated to a base pressure of 5 Torr. After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 100 Torr for 45 minutes after which point the chamber was brought to atmosphere with argon gas. The spore ampoules were removed from the enclosure using well-established sterile techniques. The stainless-steel discs were tested for SAL by gently crushing the ampoule and then incubating the ampoule for 24 hours at 60° C. for 24 hours. After a time, it was found that the discs achieved an acceptable SAL of 10-6 spores/unit.

### Example 5

A performic acid sterilant solution was generated by mixing an aqueous solution of hydrogen peroxide, 30 weight % (Sigma Aldrich, St. Louis, Mo.) with formic acid, 98-100% (Sigma Aldrich, St. Louis, Mo.) and a catalytic amount of sulfuric acid, 99.999% (Sigma Aldrich, St. Louis, Mo.). The performic acid sterilant solution was stirred at 200 RPM for 15 minutes.

The performic acid sterilant vapor was tested for its microbicidal effectiveness against Geobacillus stearothermophilus spores in a small fermentation vessel using the system described in the disclosure. Multiple self-contained biological spore ampoules (Mesa Laboratories, Bozeman, Mont.) containing a stainless-steel disc inoculated with a spore population of 106 spores/unit were placed at multiple locations within the enclosure. The enclosure was evacuated to a base pressure of 100 Torr. After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 350 Torr for 120 minutes after which point the chamber was brought to atmosphere with argon gas. The spore ampoules were removed from the enclosure using well-established sterile techniques. The stainless-steel discs were tested for SAL by gently crushing the ampoule and then incubating the ampoule for 24 hours at 60° C. for 24 hours. After a time, it was found that the discs achieved an acceptable SAL of 10-6 spores/unit.

### Example 6

A performic acid sterilant solution was generated by mixing an aqueous solution of hydrogen peroxide, 30 weight % (Sigma Aldrich, St. Louis, Mo.) with formic acid, 98-100% (Sigma Aldrich, St. Louis, Mo.) and a catalytic amount of sulfuric acid, 99.999% (Sigma Aldrich, St. Louis, Mo.). The performic acid sterilant solution was stirred at 200 RPM for 15 minutes.

The performic acid sterilant vapor was tested for its microbicidal effectiveness against Geobacillus stearothermophilus spores in a large fermentation vessel using the system described in the disclosure. Multiple self-contained biological spore ampoules (Mesa Laboratories, Bozeman, Mont.) containing a stainless-steel disc inoculated with a spore population of 106 spores/unit were placed at multiple locations within the enclosure. The enclosure was evacuated to a base pressure of 100 Torr. After reaching the base pressure, the sterilant chamber containing the sterilant solution was opened after which point the sterilant vapor was outgassed and injected into the enclosure. Heat of 40° C. was evenly applied to the sterilant chamber and to the enclosure. The system was allowed to remain in a steady state of approximately 350 Torr for 30 minutes after which point the chamber was brought to atmosphere with argon gas. The spore ampoules were removed from the enclosure using well-established sterile techniques. The stainless-steel discs were tested for SAL by gently crushing the ampoule and then incubating the ampoule for 24 hours at 60° C. for 24 hours. After a time, it was found that the discs achieved an acceptable SAL of 10-6 spores/unit.

The foregoing description has been presented for purposes of illustration and description. Furthermore, the description is not intended to limit the inventions and/or aspects of the inventions to the forms disclosed herein. Consequently, variations and modifications commensurate with the above teachings, and skill and knowledge of the relevant art, are within the scope of the presented inventions. The embodiments described hereinabove are further intended to explain best modes known of practicing the inventions and to enable others skilled in the art to utilize the inventions in such, or other embodiments and with various modifications required by the particular application(s) or use(s) of the presented inventions. It is intended that the appended claims be construed to include alternative embodiments to the extent permitted by the prior art.

What is claimed is:

1. A system for sterilizing or sanitizing an enclosed vessel, the system comprising:
a reservoir configured to hold a supply of sterilant;
a first fluid conduit having a first end fluidly connected to the reservoir and a second end fluidly connectable with an interior of an enclosed vessel;
a first valve disposed within the first fluid conduit for selectively opening and closing the first fluid conduit;
a depressurization pump;
a second fluid conduit having a first end fluidly connected to the depressurization pump and a second end fluidly connectable with said interior of said enclosed vessel; and
a controller configured to:
operate the depressurization pump to depressurize said interior of said enclosed vessel to a predetermined pressure below atmospheric pressure; and
open the first valve to expose sterilant within the reservoir to said interior of said enclosed vessel, wherein the sterilant vaporizes and expands to sterilize or sanitize interior surfaces of said enclosed vessel.

2. The system of claim 1, further comprising:
a sterilant pump configured to pump sterilant from the reservoir through the first fluid conduit.

3. The system of claim 2, wherein the controller is configured to operate the sterilant pump to inject sterilant into said interior of said enclosed vessel.

4. The system of claim 2, wherein the sterilant pump is configured to pump liquid sterilant through the first fluid conduit, wherein the liquid sterilant vaporizes within the enclosed vessel.

5. The system of claim 2, wherein the sterilant pump is configured to pump vaporized sterilant through the first fluid conduit.

6. The system of claim 2, wherein the sterilant pump comprises a vacuum pump configured to reduce a pressure within the reservoir below atmospheric pressure, wherein the vacuum pump exhausts the vaporized sterilant into the first fluid conduit.

7. The system of claim 1, further comprising:
a heater configured to heat the first reservoir.

8. The system of claim 1, further comprising:
at least one sensor for monitoring temperature or humidity within the second fluid conduit, the sensor configured to generate an output for receipt by the controller.

9. The system of claim 1, further comprising:
a carrier gas source, wherein the carrier gas source is selectively connectable to at least one of:
said interior of said enclosed vessel;
the first reservoir; and
the first fluid conduit.

10. The system of claim 9, further comprising:
a heater configured to heat the carrier gas.

11. The system of claim 1, wherein the controller operates the depressurization pump and the sterilant pump in an automated procedure.

12. The system of claim 1, further comprising:
a second valve configured to open and close the second conduit.

13. The system of claim 12, wherein the first and second valve are each connected to and operated by the controller.

14. The system of claim 1, wherein at least a portion of the first fluid conduit and a portion of the second fluid conduit share a common fluid conduit.

15. A method for sterilizing or sanitizing an enclosed vessel, the method comprising:
reducing a pressure within an interior of the enclosed vessel to a predetermined vacuum pressure below an ambient atmospheric pressure;
after reducing the pressure within the enclosed vessel, opening a fluid path between the interior of the enclosed vessel and a supply of sterilant, wherein the sterilant is exposed to the vacuum pressure;
vaporizing the sterilant; and
permitting vaporized sterilant to expand into the interior of the enclosed vessel.

16. The method of claim 15, further comprising:
heating the sterilant to a predetermined temperature prior to exposing the sterilant to the vacuum pressure.

17. The method of claim 15, further comprising:
admixing at least first and second components of the sterilant prior to exposing the sterilant to the vacuum pressure.

18. The method of claim 15, further comprising:
pumping the sterilant through the fluid path after opening the fluid path.

19. The method of claim 18, further comprising:
combining the sterilant with a carrier gas.
